(19) United States
(12) Patent Application Publication
(10) Pub. No.: US 2012/0184052 A1
(43) Pub. Date: Jul. 19, 2012

(54) Isolation and Analysis of Thiol Protein Matter Using Gold Nano-Particles

(75) Inventors: Xueji Zhang, Sarasota, FL (US); Bulent Mutus, Windsor (CA)

(73) Assignee: WORLD PRECISION INSTRUMENTS INC., Sarasota, FL (US)

(21) Appl. No.: 13/497,306

(22) PCT Filed: Sep. 28, 2010
(86) PCT No.: PCT/US10/50462

§ 371 Date: Mar. 21, 2012

Related U.S. Application Data
(60) Provisional application No. 61/246,270, filed on Sep. 28, 2009.

(51) Int. Cl. G01N 33/553; G01N 30/00

(52) U.S. Cl. 436/525; 422/69

(57) ABSTRACT

A method of rapidly and accurately identifying and analyzing thiol proteins in a sample using gold nanoparticles. Disclosed are embodiments of a flow device for isolation, fractionation and subsequent instrumental analysis of thiol containing proteins from various samples or tissue sources using gold nanoparticles. Also disclosed are embodiments of a flow device for detecting, isolating and fractionating S-nitrosated proteins and peptides for subsequent analysis, including identification of S-nitrosation sites.

### CROSS REFERENCE TO RELATED APPLICATION

This application claims the benefit of U.S. Provisional Patent Application No. 60/246,270, filed Sep. 28, 2009, the content of which is incorporated herein by reference in its entirety.

### BACKGROUND

Reactive oxygen species (ROS) including hydrogen peroxide (H₂O₂), superoxide (O₂.⁻) hydroxyl radical (OH.) and reactive nitrogen species (RNS) including nitric oxide (NO) and related oxygenated nitrogen species (NOₓ) can be produced in vivo under a variety of conditions including metabolism, proliferation, inflammation, and senescence. ROS and RNS can oxidatively modify protein thiols—on cysteine residues—thereby serve to regulate physiological events or trigger a variety of pathologies. The proteins that have redox-sensitive cysteines will be referred here as the thiol proteome.

S-nitrosoproteome is a subclass of the thiol proteome that becomes reversibly modified by NO or its derivatives (NOₓ) to yield S-nitrosated (R—S—N═O) proteins. The identification of the S-nitrosoproteome is essential in deciphering the physiological and pathophysiological consequences of NO-mediated signaling.

The identification of the thiol proteome and the regulatory pathways they affect is of primary importance since many of the proteins with redox sensitive thiols identified thus far are central to cellular function such as transcription factors, signal transducers, apoptosis and stress response proteins.

It is also known that NO can modulate cellular signaling, not only through the guanylate cyclase/cGMP pathway, but also by S-nitrosating protein and small molecular weight thiols. A large volume of subsequent work has implicated S-nitrosation of proteins in the regulation, subcellular compartmentalization and degradation of proteins. To date, ˜500 plant animal and prokaryotic thiol proteins have been identified as potential S-nitrosation targets. Therefore, the identification of the S-nitrosoproteome is an area of interest from the point of view of elucidation of RNS-mediated signaling pathways in health and disease.

S-nitrosylation of amino acid residues of proteins (in particular, cysteine) can play a significant role in numerous cellular processes including vasodilation, neurotransmission, cellular localization, and the cell cycle. This recently discovered post-translational modification also serves to store and transport nitric oxide (NO) in the form of S-nitrosothiol (SNO). Therefore, identification of sites of S-nitrosylation provides significant insight into the location, stability and frequency of these modifications.

Mass spectrometry (MS) is a common method used to determine protein modifications. One MS technique used herein is known as matrix-assisted laser desorption ionization time-of-flight MS (i.e., MALDI-TOF MS). MALDI-TOF MS is designed typically for use in protein/peptide analysis, but is occasionally limited by sample complexity, poor sample ionization and/or peak suppression.

The identification of ROS or RNS modified thiols on proteins pose many challenges since the proteins of interest are present in low μM to nM levels and the target thiols are transiently modified owing to the lability of the products.

The vast majority of the methods currently employed for detecting the thiol proteome and S-nitrosoproteome are gel electrophoresis-based. For example, diagonal electrophoresis methods have long been used to detect intra- or intermolecular disulfide bridges by performing sequential reducing and nonreducing electrophoresis, as disclosed by Brennan, J. P. et al., Detection and mapping of widespread intermolecular protein disulfide formation during cardiac oxidative stress using proteomics with diagonal electrophoresis, J Biol Chem 279: 41352-41360, 2004.

It is known that Protein glutathionylation can be detected by two-dimensional electrophoretic (2-DE) methods by taking advantage of the availability of specific antibodies. Drawbacks to these antibody-based methods include moderate resolution, nonspecific detection, and low throughput.

Characteristics of S-nitrosated proteins, such as photosensitivity and sensitivity to reduction by ascorbate, thiol reducing agents and Cu (I) complicate their isolation and detection. In contrast, the identification of the phospho proteome is significantly simpler because phosphorylated amino acids can be identified more easily by radiolabelling (³²P) or by antibodies against the phosphoamino acids. Unfortunately, radioactive N or O atoms do not exist and the few monoclonal or polyclonal antibodies that were raised against the S-nitrosocysteine have yet to be proven useful in proteomic studies.

Progress in the identification of the S-nitrosoproteome was made by the introduction by the “biotin-switch” assay by Jaffrey, S. R. et al., Protein S-nitrosylation: a physiological signal for neuronal nitric oxide, Nat Cell Biol 3:193-197, 2001. In this procedure, the free thiols are blocked with a thiol-specific methylating agent which does not disrupt nitrosothiols and disulfide bonds. Protein mixtures must be denatured to ensure that all thiols are blocked.

The protein S-nitrosothiols are subsequently reduced to thiols with ascorbate to yield thiols. In the final step, these newly formed thiols are reacted with a sulfhydryl-specific biotinylating agent. The biotinylated proteins that contained S-nitrosogroups are then detected on Western blots following incubation with streptavidin or by antibodies against biotin. A drawback to the biotin shift assay is that the ascorbate used to reduce S-nitrosothiols can also reduce disulfides other cysteine oxidation derived modifications such as S-glutathionylation or S-oxidations, including sulfenic, sulfinic, and sulfonic acids, thus producing false positives. Additional shortcomings are related to sensitivity arising from the incomplete reduction of trace levels of RSNOs and the factors that regulate the efficiency of the detection of the biotinylated proteins on the Western blots.

Saturation-labeling differential gel electrophoresis (DIGE) methods employing cysteine-specific fluorochromes have been developed to identify both the thiol proteome and S-nitrosoproteome. The DIGE approach employs a pair of fluorescent maleimide-conjugated cyanine dyes (Cy3-green fluorescence and Cy5-red fluorescence) to specifically label free thiols in multiple protein samples. Mixtures of differentially labeled proteins are run on the same gel, and computer-aided analysis of the difference in intensity between the red and green fluorescence gives the amount of S-nitrosated (or thiolated) proteins. A drawback to this technique is that differences between red and green fluorescence is difficult to determine for proteins containing a large number of DTT-reducible disulfides.

A mass spectrometry (MS)-based method, ICAT, is another non-gel-based thiol-specific proteomic technique with multiplexing capability. Two different ICAT reagents are utilized in this technique. These reagents consist of three essential groups: a thiol-reactive group, an isotope-coded light or heavy linker, and a biotin moiety for peptide isolation. In an ICAT experiment, protein samples from differing redox environments are first labeled with either light or heavy ICAT reagents. The mixtures of labeled proteins are digested and separated by chromatography. Peptides are identified by tandem MS, and the relative amounts of the peptides are inferred from the integrated LC peak areas of the heavy and light versions of the ICAT-labeled peptides.

There is a need for new robust methods and techniques for analyzing thiol and S-nitroso-proteomes.

### SUMMARY

Novel gold nanoparticle-based methods and devices for identifying the thiol proteome and S-nitrosoproteome accurately and rapidly with sensitivity capable of detecting these species at biologically relevant concentrations are disclosed.

The silicon-bound gold nanoparticles are optimized with respect to the binding of protein thiols as a function of: i) thiol oxidation state, ii) proteins size and iii) thiol accessibility.

Embodiments of flow devices for isolation, fractionation and subsequent mass spectroscopy analysis of thiol containing proteins from various tissue sources are disclosed.

Also disclosed are embodiments of flow devices for detecting, isolating and fractionating S-nitrosated proteins and peptides for subsequent analysis.

### BRIEF DESCRIPTION OF THE DRAWINGS

FIG. 1A is a graph depicting the amount of nitric oxide (NO) released as a function of gold nanoparticle (AuNP) solution concentration;

FIG. 1B is a graph depicting the amount of NO released as a function of concentration of GSNO added to AuNP solution;

FIG. 2 is a gel showing isolation of thiol proteome from various sources;

FIG. 3A is a diagram of a reaction showing NO released from a reaction of SNOBSA with AuNP according to an aspect of the disclosure;

FIG. 3B is a graph depicting the amount of NO released as a function of SNOBSA injected according to the experimental depicted in FIG. 3A;

FIG. 4 depicts an embodiment of an array employed according to the disclosure used to determine the relationship between AuNP surface area and the size of proteins that can be trapped;

FIG. 5A depicts three cysteine (C) residues for experimental use in embodiments of the disclosed method and device;

FIG. 5B depicts the results of an experimental using the cysteine (C) residues of FIG. 5A;

FIG. 6 shows a PDMS flow device employed for isolation, fractionation and identification of thiol containing proteins according to the disclosure;

FIG. 7A shows a technique of proteolysis of AuNP bound thiol proteins prior to release of thiol-peptides by reacting with sodium hydrosulfide (NaSH);

FIG. 7B shows an alternative to the technique of FIG. 7A, wherein a peptide is directly volatilized with a laser pulse;

FIG. 8 depicts a microfluidic device for practicing the disclosed method; and

FIG. 9 depicts an alternative embodiment of a device for practicing the disclosed method.

### DETAILED DESCRIPTION

Gold nanoparticles (AuNPs) are useful as tools in many areas of research owing to their high affinity towards thiols, distinctive optical properties and ease of isolation.

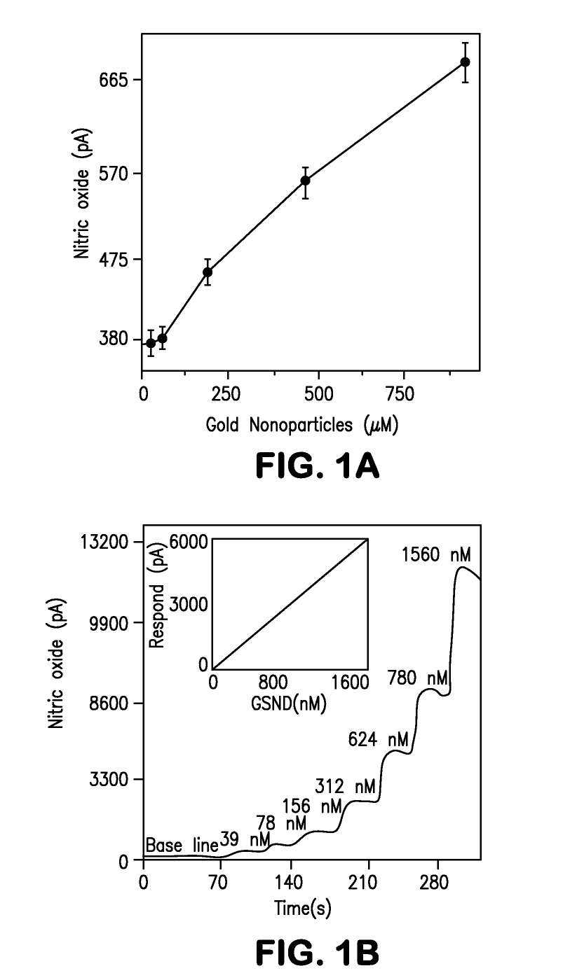

It has recently been discovered that gold nanoparticles (AuNP) can denitrosate (i.e., release NO from) small molecular weight and protein S-nitrosothiols (see FIG. 1) yielding gold thiolates according to Scheme I:

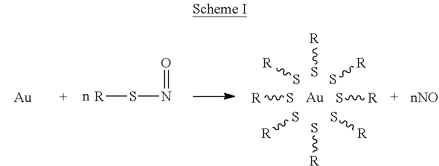

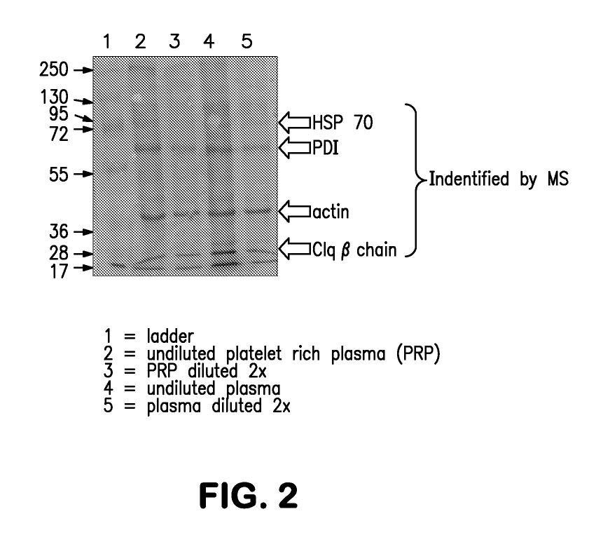

It has been shown that AuNP can be utilized for the rapid and facile isolation of thiol proteome from serum or cellular fractions (see FIG. 2). In these experiments, AuNP were added directly into plasma or platelet rich plasma. The AuNP were harvested by centrifugation and further purified by gel chromatography. The proteins attached to the AuNP were then released by subsequent exposure of the AuNP-protein complexes to large concentrations of dithiothreitol (DTT) or NaSH, followed by exposure to sodium dodecyl sulfate polyacrylamide gel electrophoresis (SDS-PAGE). A limited number of these proteins have been identified by MS. The same thiol proteins (FIG. 2) were reproducibly isolated by this simple procedure.

It was recently discovered that AuNP covalently linked to silicon can be synthesized on poly(dimethylsiloxane) (PDMS) surfaces. PDMS is a widely employed polymer material for fabricating fluidic devices.

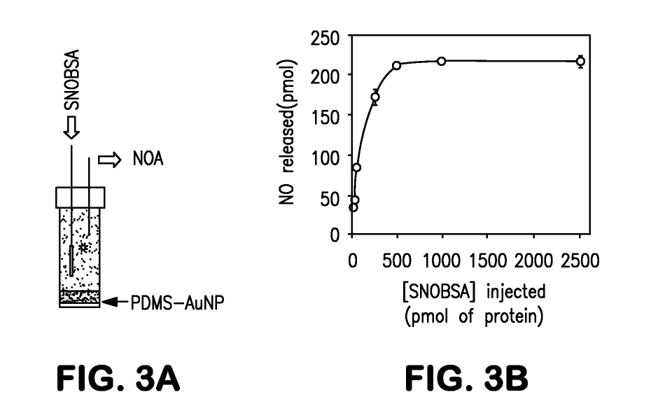

PDMS bound AuNP has been shown to effectively denitrosate S-nitroso-bovine serum albumin (SNOBSA). In a known method, 1 mm layer of PDMS-AuNP (˜10 nm diameter) with a surface area of ˜0.25 cm² surface was created at the bottom of septa capped HPLC sampling vials (FIG. 3a). Increasing amounts of SNOBSA were added into the vial. A sample (200 μL) of the gas phase above the surface was injected into a GE-Sievers nitric oxide analyzer (NOA). The results indicate that the ˜0.25 cm² PDMS-AuNP surface has a capacity to form ˜200 μmol of BSA-goldthiolate and release ˜200 μmol NO in the process, according to Scheme I.

Embodiments of a method and instrument for detecting, isolating and analyzing thiol proteins using gold nano-particles are disclosed.

### Methods

A significant component of the disclosed fluidic device is the gold nanoparticles (AuNPs) immobilized on the PDMS surface for the capture of the thiol proteome or the S-nitrosoproteome. The AuNP react with free thiols (R—SH), S-nitrosothiols (R—S—N═O) and/or disulfides (R—S—S—R′) to yield Au-thiolates. These functionalities can be thus captured on proteins. The size and surface ligand composition of the PDMS-bound AuNP can be varied to prepare a PDMS-bound AuNP surface that can accommodate binding proteins of varying sizes, including proteins containing thiol, disulfide and S-nitrosothiol groups.

The data presented herein is incorporated into the design of a flow device that interacts efficiently with the thiol proteome. The thiol proteome are subsequently fractionated off of the PDMS-bound AuNP by increasing thiol gradients and analyzed.

The PDMS-bound AuNP surface affords specificity for the detection of S—NO containing proteins since these proteins release NO upon interacting with AuNP to yield Au-Protein thiolates. Several embodiments of a fluidic device can be employed for detecting SNO-proteins via incorporation of a sensitive NO-detection system into the reaction chamber where monodisperse or PDMS-bound AuNP come into contact with protein fractions.

The efficiency of binding of silicon-bound gold nanoparticles with protein thiols changes as a function of thiol oxidation state, protein-size and thiol accessibility. AuNPs interact with numerous functional groups including amine, hydroxyl, carboxylate, vinyl and aromatic, without limitation. However, it is known that the largest association constants are with thiols (see, for example, Marcon, L. et al., Characterization of nanogap chemical reactivity using peptide-capped gold nanoparticles and electrical detection, Bioconjug Chem., 19:802-805, 2008.). Therefore, interactions of non-thiol functional groups with AuNP can be eliminated by first covering the AuNP with thiol-containing ligands. First, the type and size of thiol-ligand (S—R) that is most effectively exchanged by protein thiols according to Scheme II is determined:

Protein-SH+PDMS-AuNP—S—R→PDMS-AuNP—S-Protein+HS—R Scheme II

AuNP can also bind small molecular weight disulfides and reduce them on their surfaces. It may also be possible for protein-mixed disulfides (Protein-S—SR) can also exchange with AuNP-bound thiolates and be reduced in the process, as outlined in Scheme III:

Protein-S—SR′+PDMS-AuNP—S—R PDMS-AuNP—S-Protein+HS—R Scheme III

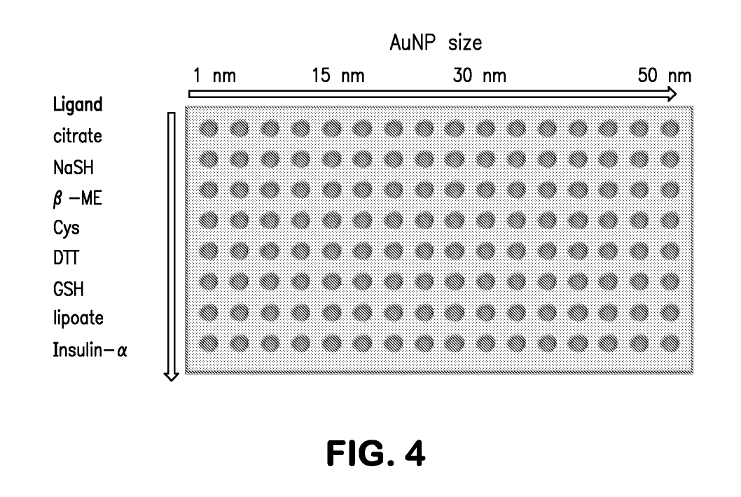

The disclosed method also stresses the determination of the relationship between AuNP surface area and the size of proteins that can be trapped. As shown in FIG. 4, arrays of varying AuNP size vs. Au-bound ligand are employed for this determination.

The optimal conditions on PDMS-bound AuNP for trapping the thiol proteome is determined by the following process. The size of AuNP on PDMS surface is controlled by regulating the incubation time and [AuHCl₄], as described in Zhang, Q., et al., In-situ synthesis of poly(dimethylsiloxane)-gold nanoparticles composite films and its application in microfluidic systems, Lab on a Chip 8:352-357; 2007. Arrays of PDMS-bound AuNP vs. Au-bound ligand are constructed, as depicted in FIG. 4. In this embodiment, the ligands tested include citrate (control ligand) and thiols of increasing molecular weight—sodium hydrosulfide (NaSH), β-ME, Cysteine (Cys), Dithiothreitol (DTT), Glutathione (GSH), lipoate and insulin-α. By decorating the Au surface with thiols—the ligands with the highest affinity for Au—binding of all weaker non-thiol interactions is eliminated.

The ligands with the smallest molecular weight typically bind most strongly to the AuNP surface. The small thiol ligands are therefore most efficiently exchanged by small molecular weight thiol components of biological samples. Proteins are thus displaced most efficiently by the weaker-bound larger peptide thiols.

Arrays with varying AuNP size and Au-bound ligands, such as those depicted in FIG. 4, are subsequently exposed to serum proteins. Each spot in the array is washed free of non-specifically adhered molecules, leaving behind thiols bound to AuNP surface. The bound thiols are eluted by excess NaSH and subsequently identified by mass spectroscopy.

The ability of the AuNP to bind to mixed disulfide containing proteins by converting proteins that are known to be susceptible to mixed disulfide formation to their glutathionyl derivatives can be similarly determined. The mixed disulfide containing proteins are first exposed to excess glutathione disulfide (GSSG) and tested like the above thiols to determine AuNP binding efficiency.

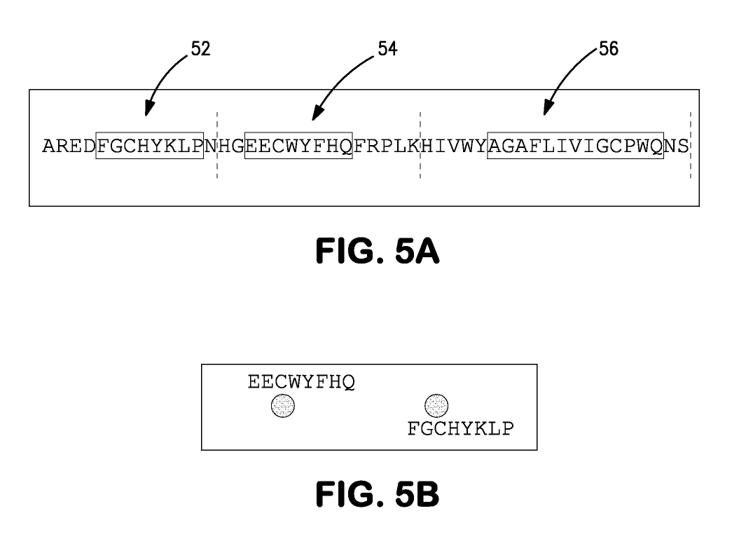

Once the preferred combination of AuNP size surface and ligand for trapping a wide range of thiol proteins is determined, the type of thiols—surface resident, partially buried or buried—that are best trapped by the AuNP is identified. A variety of thiol containing proteins with known 3D structures are applied onto the AuNP, as illustrated in FIGS. 5A and 5B. A protein of known 3D structure has three cysteine (C) residues. One is solvent exposed (52), a second is partially buried (54), and a third is fully buried (56), as shown in FIG. 5A. The protein is exposed to S-nitrosation conditions and applied onto polydimethylsiloxane (PDMS)-AuNP. The AuNP-bound protein is subsequently proteolyzed and the bound-thiol peptides identified by MS to yield the sequences EECWYFHQ and FGCHYKLP, as shown in FIG. 5B. This result indicates that the AuNP can interact with solvent exposed (54) and partially buried thiols (52), and not fully buried thiols 56.

Another method of determining accessibility of the AuNP to S-nitrosated cysteines is by measuring the amount of nitric oxide (NO) released upon interacting with the AuNP-surface, as shown in Scheme I. Again using the protein shown in FIG. 5A, both the EECWYFHQ and FGCHYKLP cysteines can be modified by S-nitrosation. Under such conditions, approximately 2 moles of NO will be released per mole of protein. NO release is monitored upon exposure to the AuNP surface and again upon addition of copper ions (Cu⁺) which have been shown to release NO from solvent inaccessible S-nitrosated cysteine residues. The accessibility of the AuNP to various thiol environments and domains in proteins is mapped accordingly.

### Instrumentation

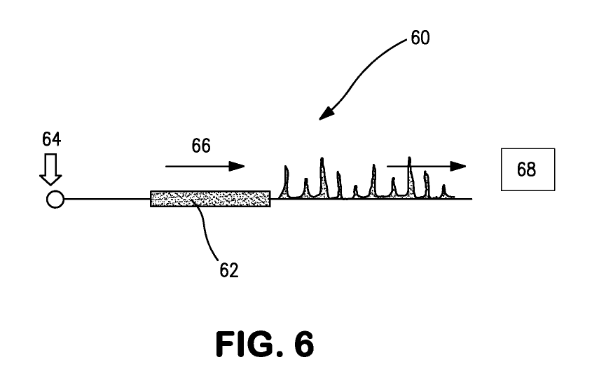

A variety of embodiments of flow devices for isolation, fractionation and subsequent identification of thiol containing proteins from various tissue sources according to the disclosure exist. An embodiment which utilizes PDMS is depicted as reference numeral 60 in FIG. 6. Of course the embodiment is not limited to PDMS; any inert material known in the art can be substituted. A microchannel network within the PDMS bound-AuNP region 62 can optionally be employed to increase the overall surface area of PDMS bound-AuNP. The optimal size and surface-liganding of the AuNP attached to this region for thiol proteome trapping is typically determined experimentally, as detailed above. The thiol proteome first interacts with the AuNP surface 62 as the sample 64 is injected into the device. The bound thiol proteins are subsequently fractionated off of the AuNP surface 62 by eluting with gradients of increasing concentration of a small thiol 66, such as NaSH.

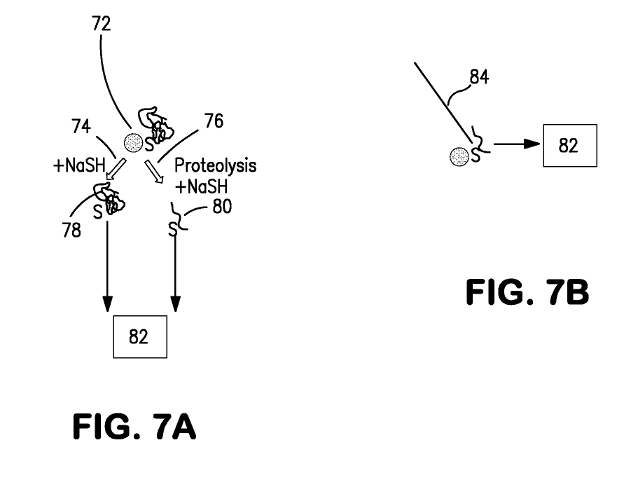

Several small molecular thiols are typically utilized to determine efficiency for eluting the particular bound proteins. The collected protein fractions are subsequently identified by mass spectroscopy (MS) 68. Alternatively, the bound thiol proteins can first be subjected to proteolysis, followed by eluting with thiol gradients, as discussed above and represented in FIG. 7A.

A variety of additional embodiments of flow devices for detecting, isolating and fractionating S-nitrosated proteins and peptides for subsequent mass spectroscopy analysis according to the disclosure exist. In one such embodiment, fractions of a proteome are introduced into multi-well plates, the bottoms of which are coated with a relatively thin layer of PDMS-AuNP. The S-nitrosated fractions release NO upon making contact with the AuNP surface and the protein bonds to the AuNP via its denitrosated thiolate. The NO released is detected by the NO-electrode and the well is identified as containing an S-nitrosated protein. Subsequently, there are two options with respect to the AuNP-bound S-nitrosated protein, shown generally in FIG. 7A—(1) the protein 78 can first be released from the AuNP surface by contacting with a small molecular weight thiol (for example, NaSH; see 74 in FIG. 7A), followed by subjecting to proteolysis and MS identification 82 of the protein from its proteolytic digestion patters; or (2) the protein bound to the AuNP can first be proteolyzed on the AuNP, followed by release of the thiol-peptide 80 by contacting with excess thiol and MS identification 82 (for example, NaSH; see 76 in FIG. 7A). The MS analysis of the thiol containing peptide will identify the S-nitrosation site in the protein. With reference to FIG. 7B, it is also possible that, subsequent to the addition of a laser absorbing matrix—for example, sinapinic acid—the thiol peptide can be directly volatilized into the MS 82 via a laser pulse 84, thus avoiding extra manipulation steps.

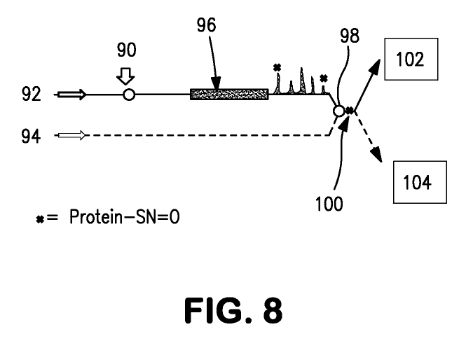

Embodiments of a flow-dependent instrument exist that utilize monodisperse AuNP. An embodiment depicted in FIG. 8 is a standalone microfluidic device. In this device, the cells or tissues to be analyzed are homogenized and the desired subcellular fraction 90 is injected into the buffer stream 92. In a parallel channel, AuNP 94 enters the device at substantially the same flow rate as the buffer channel 92. The proteins in the buffer channel 92 are then fractionated 96 according to hydrophobicity of the buffer stream and are mixed in a 1:1 ratio with AuNPs at a mixing chamber 98. Within the mixing chamber is a NO microelectrode. If the protein has a SNO group, NO release will cause the microprocessor 100 to open a valve and deliver the sample for analysis by MS 102. Conversely, when no NO is detected by the microelectrode, the microprocessor 100 causes the samples to be delivered elsewhere, for example, to a waste container 104. Fractions of the AuNP-bound, denitrosated protein are collected in this manner. This embodiment is particularly useful because the AuNP—S-protein complexes are stable and can be stored for later MS analysis, rather than immediate analysis with concern of decomposition. Prior to MS analysis, the AuNP—S-protein complexes are exposed to large concentrations of small molecular weight thiol, thus releasing the Protein-SH from the AuNP, or they are first proteolyzed in order to directly identify the thiol-bearing peptide by MS (respective procedures shown in FIG. 7A).

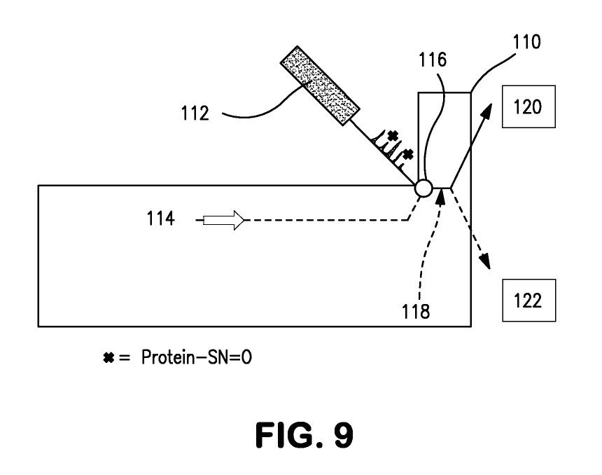

FIG. 9 depicts an additional embodiment which is a slight variation of the device of FIG. 8. Instead of being a stand alone device, the device 110 is incorporated onto the outflow on an HPLC 112. The AuNP stream 114 is maintained at substantially the same flow rate as the HPLC 112. In the mixing chamber 116, AuNP from the stream 114 reacts with the protein fractions coming off of the HPLC 112. The detection and analysis is identical to the stand alone device of FIG. 8, with microprocessor 118 causing the protein-SH to be delivered to the MS 120 for analysis or the sample to be delivered elsewhere, like for example, to the waste container 124.

In employing the above methods and devices, PDMS-bound AuNP is first prepared. Particularly useful preparation techniques are presented in Chen, H-Y et al., In-situ synthesis of poly(dimethylsiloxane)-gold nanoparticles composite films and its application in microfluidic systems, Lab on a Chip, 8:352-357, 2007, where varying HAuCl₄ are incubated on a cured PDMS surface. At various time intervals the HAuCl₄ solution is washed off and the size of the PDMS-bound AuNP is determined at the TEM facilities.

The above embodiments represent a new and useful method and device for direct electrochemical measurement of S-nitrosothiols having a detection limit lower than 0.1 nM. The disclosed method allows the measurement of RSNO in real time with high sensitivity levels.

Many known NO sensors having high sensitivity and exhibit significant temperature sensitivity in the low nanomolar NO concentration range. This phenomenon limits their usefulness in many applications involving in vivo RSNO measurements at NO concentration levels anticipated to be less than 10 nM. The disclosed methods and instruments preferably employ a new generation of NO sensors with low temperature coefficients and detection limits of 0.1 nM based on nanotechnology.

### EXAMPLE

Two model proteins were used in this example—protein disulfide isomerase (PDI) and dual specificity phosphatase 12 (hYVH1).

Reduced hYVH1 was S-nitrosylated by treatment with S-nitrosoglutathione (GSNO). Any remaining free thiols were then S-carbamidomethylated (CM) by addition of iodoacetamide (IAM). The SNO was then selectively reduced to SH with ascorbate, which does not reduce disulfides. The newly formed SH was then differentially alkylated with N-ethylmaleimide (NEM), yielding previously free thiols as S-CM and SNO as S-NEM.

A separate batch of hYVH1 was S-glutathionylated by addition of oxidized glutathione (GSSG) and any remaining free thiols were S-carbamidomethylated by addition of IAM. This batch was pooled with the differentially alkylated hYVH1.

MS analysis was used to determine at which sites proteins were being S-nitrosylated. Fully alkylated protein was compared to the spectrum of protein that had been first nitrosylated, followed by alkylation. Due to the similarity in absorption energy of the SNO bond (335 nm) and the emission energy of the MALDI laser (317 nm), SNO dissociates upon irradiation with the laser and appeared as free thiol in the resultant mass spectrum.

The proteins were then subjected to proteolytic digestion and the resulting peptide pool was added to the AuNPs. After incubation, the AuNPs were centrifuged, washed and the bound peptides were eluted by addition of dithiothreitol (DTT). The resulting eluant was collected, desalted and analyzed by MS (specifically MALDI-TOF MS). It should be noted that elution with DTT reduces the mixed disulfide formed by S-glutathionylation, meaning that free thiols that appear in the mass spectrum existed as SSG peptides prior to elution by DTT.

Using the differential alkylation technique combined with MS analysis, it is clear that AuNP enrich thiol-containing peptides regardless of whether the thiols were unmodified, alkylated or thioethers (i.e. methionine). These results allow the determination of which protein sites were unmodified, S-nitrosylated, or S-glutathionylated.

AuNP can effectively enrich thiol-containing peptides from a pool of proteolytic peptides, thus simplifying the resulting mass spectrum as well as increasing the relative intensities of the peaks of interest. This simplification and amplification allows for the unambiguous identification of sites of S-nitrosylation and S-glutathionylation in a single experiment.

Further, upon comparison of mass spectra of alkylated and non-alkylated S-nitrosylated proteins, it became apparent that S-nitrosylation affords some protection against alkylation.

For both model proteins, the saturating peaks in the mass fingerprints were non-thiol-containing. After AuNP enrichment however, the saturating peaks were thiol-containing peptides for both proteins proving that AuNP were able to enrich thiol-containing peptides. Enrichment also significantly increases the relative peak intensities of other non-saturating thiol-containing peptides, thus facilitating unambiguous identification by post-source decay (PSD) analysis.

The AuNP are somewhat large in comparison to protein microenvironments (i.e., approximately 11 nm±1 nm). This property can lead to a belief that steric interference of amino acid side chains would hinder the interaction of AuNP with intra or intermolecular protein disulfides. The a′ domain of PDI contains a pair of vicinal cysteines that have been shown to be susceptible to formation of a disulfide more so than the a domain bearing the same motif. When subjected to AuNP enrichment, the a′ peptide no longer appears in the mass fingerprint suggesting that AuNPs do not interact with disulfides.

When hYVH1 was S-nitrosylated, S-glutathionylated, S-carbamidomethylated and S—N-ethylsuccinimidylated, AuNP demonstrated enrichment for all thiol-containing peptides, including methionine. Sites of S-nitrosylation and S-glutathionylation were subsequently identified by PSD analysis of individual peptides.

These results establish that thiols and thioethers have a greater affinity for gold than other protein functional groups, even after chemical modification by, for example, alkylation or thiolation. Exploitation of this property is the basis for the disclosed simple, novel method for isolating, detecting and enriching S-modified peptides in a single step using AuNP. When combined with thiol modification and mass spectrometry, the disclosed AuNP-based method can unambiguously identify sites of protein modification.

While preferred embodiments have been shown and described, various modifications and substitutions may be made thereto without departing from the spirit and scope of the invention. Accordingly, it is to be understood that the disclosed embodiments have been described by way of illustration and not limitation.

What is claimed:

1. A method of identifying and analyzing thiol proteins, comprising the steps of:
providing a source of gold nanoparticles;
contacting said gold nanoparticles with a sample comprising one or more thiol proteins to yield gold-bound thiol proteins;
eluting said gold-bound thiol proteins with an eluting solution capable of releasing said thiol proteins from said gold nanoparticles; and
identifying said eluted thiol proteins by a suitable analytical method.

2. The method of claim 1, wherein said eluting solution comprises at least one thiol with a higher affinity for gold nanoparticles than said thiol protein.

3. The method of claim 1, wherein said thiol protein has at least one cysteine residue.

4. The method of claim 1, wherein said thiol protein is an S-nitrosated protein.

5. The method of claim 4, comprising the step of detecting nitric oxide released from the reaction of said S-nitrosated protein and said gold nanoparticles.

6. The method of claim 4, wherein said S-nitrosated protein has at least one cysteine residue.

7. The method of claim 4, comprising the step of proteolyzing the gold nanoparticle bound S-nitrosated protein prior to eluting said gold-bound protein to yield a thiol peptide, and the step of identifying the S-nitrosation site in the protein via analysis of said thiol peptide via a suitable analytical technique.

8. The method of claim 4, comprising the step of proteolyzing the thiol protein after releasing said protein from said gold nanoparticles to yield a thiol peptide, and identifying the S-nitrosation site in the protein via analysis of said thiol peptide via a suitable analytical technique.

9. The method of claim 1, wherein said suitable analytical method is mass spectrometry.

10. The method of claim 7, wherein said suitable analytical method is mass spectrometry.

11. The method of claim 8, wherein said suitable analytical method is mass spectrometry.

12. The method of claim 1, comprising the step of contacting said gold nanoparticles with at least one thiol-containing ligand prior to contacting said gold nanoparticles with said sample comprising one or more thiol proteins.

13. A device for practicing the method of claim 1, comprising:
a flow line extending from an inlet to an outlet; and
a source of gold nanoparticles positioned between said inlet and said outlet; wherein
a sample comprising one or more thiol proteins is injected into said inlet and contacts said source of gold nanoparticles, and an eluting solution is subsequently passed through said flow line.

14. The device of claim 13, wherein said outlet leads to a suitable analytical instrument for identifying said protein.

15. The device of claim 13, comprising a region of a generally inert polymeric material within which said gold nanoparticles are bound.

16. The device of claim 15, wherein said polymeric material is polydimethylsiloxane (PDMS).

17. A device for practicing the method of claim 5, comprising:
a fluidic buffer channel having an inlet and extending to a mixing chamber for transporting a sample from said inlet to said mixing chamber;
a second fluidic channel extending from a second inlet to said mixing chamber for transporting a source of gold nanoparticles from said second inlet to said mixing chamber; and
an electrode positioned within said mixing chamber for detecting nitric oxide therewithin; wherein
a sample comprising one or more S-nitrosated thiol proteins is injected into said buffer channel inlet and reacts with said gold nanoparticles within said mixing chamber to produce nitric oxide that is detected by said electrode.
